(19) United States
(12) Patent Application Publication
(10) Pub. No.: US 2019/0211349 A1
(43) Pub. Date: Jul. 11, 2019

(54) BLACK-SPOT RESISTANT POTATOES WITH REDUCED TUBER-SPECIFIC POLYPHENOL OXIDASE ACTIVITY

(71) Applicant: CELLECTIS, Paris (FR)

(72) Inventors: Benjamin Clasen, South St. Paul, MN (US); Song Luo, Chicago, IL (US); Daniel F. Voytas, Falcon Heights, MN (US); Feng Zhang, Maple Grove, MN (US)

(21) Appl. No.: 16/326,549

(22) PCT Filed: Aug. 18, 2017
(86) PCT No.: PCT/US2017/047604

§ 371 Date: Feb. 19, 2019

Related U.S. Application Data
(60) Provisional application No. 62/489,632, filed on Apr. 25, 2017.
(60) Provisional application No. 62/376,597, filed on Aug. 18, 2016.

(51) Int. Cl. C12N 15/82; C12N 15/52

(52) U.S. Cl. CPC C12N 15/8261; C12N 9/0059; C12N 15/52

(57) ABSTRACT

Materials and methods are provided for making plants (e.g., Solanum varieties) with resistance to black-spot bruising, specifically, by making TALE-nuclease-induced mutations in genes encoding tuber specific expressed polyphenol oxidase.

### CROSS-REFERENCE TO RELATED APPLICATIONS

This application claims benefit of priority from U.S. Provisional Application No. 62/376,597, filed Aug. 18, 2016, and U.S. Provisional Application No. 62/489,632, filed on Apr. 25, 2017, both of which are incorporated herein by reference in their entirety.

### TECHNICAL FIELD

This document provides materials and methods for creating potato varieties with reduced polyphenol oxidase activity.

### BACKGROUND

Black-spot bruise is a serious problem for the potato industry, resulting in major losses to commercial potato processors that produce chips and French fries. Black-spot bruise occurs upon physical impact or following damage (e.g., mechanical damage) to tubers, which initiates enzymatic browning. Symptoms of black-spot bruise include production of black, brown, and red pigments in the potatoes.

### SUMMARY

Bruise resistance is a trait important to growers and processors alike, as reduced bruise damage can minimize crop rejection and waste in processing due to discarding of blackened fries and chips. This document provides materials and methods for creating potato varieties that have increased bruise resistance due to reduced expression of tuber-specific polyphenol oxidase (PPO), which converts monophenols to o-diphenols, and o-dihydroxyphenols to o-quinones. The potato varieties described herein can be produced without the use of transgenesis. Potato varieties having mutations in genes encoding PPO also are provided.

This document is based at least in part on the discovery that potatoes with reduced tuber-specific PPO can be obtained using a sequence-specific nuclease to make a targeted mutation or knockout in tuber-specific PPO genes, specifically POT32 and POT33. The modified potatoes have increased resistance to black-spot bruising, and their use can result in reduced levels of crop rejection and waste as compared to non-modified potatoes. Further, the potatoes do not carry any foreign DNA and therefore may be considered by regulatory agencies as non-GM. This document also is based at least in part on the development of potato cultivars with loss-of-function PPO mutations generated by sequence-specific nucleases.

In one aspect, this document features a Solanum plant, plant part, or plant cell containing a mutation in at least one polyphenol oxidase (PPO) allele endogenous to the plant, plant part, or plant cell, such that the plant, plant part, or plant cell has reduced expression of PPO as compared to a control Solanum plant, plant part, or plant cell that lacks the mutation. Each mutation can be a deletion of more than one nucleotide base pair. Each mutation can be at a target sequence as set forth in SEQ ID NO:2340 or 2341, at a target sequence having at least 95 percent identity to SEQ ID NO:2340 or 2341, at a target sequence as set forth in SEQ ID NO:2342 or 2343, or at a target sequence having at least 95 percent identity to SEQ ID NO:2342 or 2343. The mutation can have been made using a rare-cutting endonuclease. The rare-cutting endonuclease can be a transcription activator-like effector endonuclease (TALE-nuclease). The TALE-nuclease can bind to a sequence as set forth in any of SEQ ID NOS:3-14 or 2346-2363. The at least one PPO allele can exhibit removal of an endogenous nucleic acid and does not include any exogenous nucleic acid. The at least one PPO allele can be a POT32 allele or a POT33 allele. The at least one PPO allele can be one or more POT32 alleles, where the one or more POT32 alleles contain one or more sequences selected from the group consisting of SEQ ID NO:2332, SEQ ID NO:2333, and SEQ ID NO:2334, or one or more sequences selected from the group consisting of SEQ ID NO:2336, SEQ ID NO:2337, SEQ ID NO:2338, and SEQ ID NO:2339. Every endogenous allele of POT32 can be mutated, every endogenous allele of POT33 can be mutated, or every endogenous allele of both POT32 and POT33 can be mutated. Each POT32 allele, each POT33 allele, or each POT32 and POT33 allele can exhibit removal of an endogenous nucleic acid and does not include any exogenous nucleic acid. The plant, plant part, or plant cell may have no detectable expression of PPO. The Solanum plant, plant part, or plant cell can be a Solanum tuberosum plant, plant part, or plant cell. The plant, plant part, or plant cell can have decreased levels of quinones as compared to a control plant, plant part, or plant cell that lacks the mutation.

In another aspect, this document features a method for making a Solanum plant that has reduced expression of PPO. The method can include (a) contacting a population of Solanum plant cells containing at least one functional endogenous PPO allele with one or more rare-cutting endonucleases targeted to the at least one PPO allele, (b) selecting, from the population, a cell in which the at least one PPO allele has been mutated, and (c) growing the selected plant cell into a Solanum plant, where the Solanum plant has reduced expression of PPO as compared to a control Solanum plant in which the at least one PPO allele has not been mutated. The Solanum plant cells can be protoplasts. The method can include transforming the protoplasts with a nucleic acid encoding the rare-cutting endonuclease. The nucleic acid can be an mRNA. The nucleic acid can be contained within a vector. The method can include introducing into the protoplasts a rare-cutting endonuclease protein (e.g., a TALE-nuclease). The TALE-nuclease can be targeted to a sequence as set forth in SEQ ID NO: 2340 or 2341, or to a sequence having at least 95 percent identity to the sequence set forth in SEQ ID NO: 2340 or 2341. The TALE-nuclease can be targeted to a sequence as set forth in SEQ ID NO: 2342 or 2343, or to a sequence having at least 95 percent identity to the sequence set forth in SEQ ID NO: 2342 or 2343. The TALE-nuclease can bind to a sequence as set forth in any of SEQ ID NOS:3-14 or 2346-2363. The at least one PPO allele can be a POT32 allele or a POT33 allele. The at least one PPO allele can be one or more POT32 alleles, and the one or more POT32 alleles can contain one or more sequences selected from the group consisting of SEQ ID NO:2332, SEQ ID NO:2333, and SEQ ID NO:2334, or one or more sequences selected from the group consisting of SEQ ID NO:2336, SEQ ID NO:2337, SEQ ID NO:2338, and SEQ ID NO:2339. The Solanum plant cells can be protoplasts. The method can further include culturing the protoplasts to generate plant lines, and/or isolating genomic DNA comprising at least a portion of the PPO locus from the protoplasts. The Solanum plant cells can be S. tuberosum plant cells.

In another aspect, this document features a method for producing a bruising resistant potato product. The method can include (a) providing a Solanum plant or plant part that contains a mutation in at least one PPO allele endogenous to the plant or plant part, such that the plant, plant part, or plant cell has reduced expression of PPO as compared to a control Solanum plant or plant part that lacks the mutation; and (b) producing a bruising resistant potato product from the plant or plant part. The mutation can be at a target sequence as set forth in SEQ ID NO: 2340 or 2341, at a target sequence having at least 95 percent identity to SEQ ID NO: 2340 or 2341, at a target sequence as set forth in SEQ ID NO: 2342 or 2343, or at a target sequence having at least 95 percent identity to SEQ ID NO: 2342 or 2343. The mutation can have been made using a rare-cutting endonuclease (e.g., a TALE-nuclease). The TALE-nuclease can bind to a sequence as set forth in any of SEQ ID NOS: 3-14 or 2346-2363. The at least one PPO allele can be a POT32 allele or a POT33 allele. The at least one PPO allele can be one or more POT32 alleles, where the one or more POT32 alleles contain one or more sequences selected from the group consisting of SEQ ID NO:2332, SEQ ID NO:2333, and SEQ ID NO:2334, or one or more sequences selected from the group consisting of SEQ ID NO:2336, SEQ ID NO:2337, SEQ ID NO:2338, and SEQ ID NO:2339. The Solanum plant or plant part can be a S. tuberosum plant or plant part. The Solanum plant or plant part may have no detectable expression of PPO.

This document also features a bruising resistant tuber generated from a Solanum plant or plant part that contains a mutation in at least one PPO allele, such that the bruising resistant tuber has reduced expression of PPO as compared to a control Solanum plant, plant part, or plant cell that lacks the mutation. Each mutation can be a deletion of more than one nucleotide base pair. Each mutation can be at a target sequence as set forth in SEQ ID NO: 2340 or 2341, at a target sequence having at least 95 percent identity to SEQ ID NO: 2340 or 2341, at a target sequence as set forth in SEQ ID NO: 2342 or 2343, or at a target sequence having at least 95 percent identity to SEQ ID NO: 2342 or 2343. Each mutation can have been made using a rare-cutting endonuclease (e.g., a TALE-nuclease). The TALE-nuclease can bind to a sequence as set forth in any of SEQ ID NOS:3-14 or 2346-2363. The tuber can have decreased levels of quinones as compared to tubers generated from a control plant or plant part that lacks the mutation. The Solanum plant or plant part can be a S. tuberosum plant or plant part. The at least one PPO allele can be a POT32 allele or a POT33 allele. The at least one PPO allele can be one or more POT32 alleles, wherein the one or more POT32 alleles contain one or more sequences selected from the group consisting of SEQ ID NO:2332, SEQ ID NO:2333, and SEQ ID NO:2334, or one or more sequences selected from the group consisting of SEQ ID NO:2336, SEQ ID NO:2337, SEQ ID NO:2338, and SEQ ID NO:2339.

In yet another aspect, this document features a food product produced from the bruising resistant tuber provided herein. The food product can have been cooked. The food product can be a French fry, chip, crisp, potato, dehydrated potato or baked potato.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows POT32 and POT33 DNA sequences (SEQ ID NOS:1 and 2, respectively) from one allele that is targeted by PPO TALE-nucleases. The underlined or bold sequences represent target sites (SEQ ID NOS:3-14) for TALE-nucleases that recognize the expressed potato tuber-specific PPO genes. In particular, bold sequences indicate target sites for the TALE-nuclease pair POT32_T3. Underlined sequences indicate target sites for TALE-nuclease pairs POT32_T1, POT32_T2, POT33 T1, POT33 T2, and POT33 T3.

FIG. 2A shows a representative example of a naturally occurring S. tuberosum nucleotide sequence for the tuber-specific PPO, POT32 (SEQ ID NO: 15).

FIG. 2B shows a representative example of a naturally occurring S. tuberosum nucleotide sequence for the tuber-specific PPO, POT33 (SEQ ID NO: 16).

FIG. 3 shows nucleic acid sequences from two different StPOT32 alleles identified in S. tuberosum (Ranger Russet). Alleles 1 and 2 are shown in SEQ ID NOS:2340 and 2341, respectively.

FIG. 4 shows nucleic acid sequences from two different StPOT33 alleles identified in S. tuberosum (Ranger Russet). Alleles 1 and 2 are shown in SEQ ID NOS:2342 and 2343, respectively.

FIG. 5 shows sequences with deletions identified in POT32 from plant St381. The mutations included a 7 bp deletion (SEQ ID NO:2331), an 11 bp deletion (SEQ ID NO:2333), and a 75 bp deletion (SEQ ID NO:2334).

FIG. 6 shows sequences with deletions identified in POT32 from plant St465. The mutations included a 6 bp deletion (SEQ ID NO:2336), a 9 bp deletion (SEQ ID NO:2337), a 16 bp deletion (SEQ ID NO:2338), and a 46 bp deletion (SEQ ID NO:2339).

FIG. 7 is a graph plotting the level of polyphenolic-oxidase enzymatic activity in tubers from line St381.

FIG. 8 shows a POT32 protein sequence (SEQ ID NO:2344). Underlined amino acids indicate the plastid transit peptide sequences at the N-terminus of the protein. Bold amino acids indicate the two copper-binding regions, CuA and CuB.

FIG. 9 shows a POT33 protein sequence (SEQ ID NO:2345). Underlined amino acids indicate the plastid transit peptide sequences at the N-terminus of the protein. Bold amino acids indicate the two copper-binding regions, CuA and CuB.

### DETAILED DESCRIPTION

Black spot bruise occurs due to physical impact or following damage to tubers and can cause major losses to commercial potato processors that produce potato chips and French fries. Mechanical damage initiates enzymatic browning, with symptoms including production of black, brown and red pigments. Bruise resistance is a trait important to growers and processors alike, as reduced bruise damage can minimize crop rejection and waste in processing due to automatic discarding of blackened fries and chips.

The reaction leading to pigment production is catalyzed by PPO, which converts monophenols to o-diphenols and o-dihydroxyphenols to o-quinones (Vamos-Vigyazo, CRC Critical Reviews in Food Science and Nutrition 14:44, 1981). PPO in potato is encoded by a gene family of at least six genes, including POTP1 and POTP2 (Hunt et al., Plant Mol Biol 21:59-68, 1993), as well as POT32, POT33, POT41 and POT72 (Thygesen et al., Plant Physiology 109:525-531, 1995). The genes are differentially expressed, with POT32 being the major form expressed in tubers. POT33 and POT72 also are expressed in tubers, while POTP1 and POTP2 are mainly expressed in leaves and flowers (Thygesen et al., supra). Differential tissue-specific expression of PPO genes also has been observed in tomato (Newman et al., Plant Mol Biol, 21: 1035-1051, 1993). The tomato PPO genes share greater identity with their interspecific homolog (tomato-potato) than with the other intraspecific PPO genes.

Thus, POT32 is implicated as a candidate gene for enzymatic discoloration. This document provides potato plant varieties, particularly of the species S. tuberosum, which have reduced PPO activity or even lack tuber PPO activity, as well as methods for generating such plant varieties. Methods for using such plant varieties (e.g., by the food industry) to produce bruising resistant potato products also are provided.

As used herein, the terms “plant” and “plant part” refer to cells, tissues, organs, seeds, and severed parts (e.g., roots, leaves, and flowers) that retain the distinguishing characteristics of the parent plant. “Tuber” refers to a thickened underground part of a stem or rhizome, serving as a food reserve and bearing buds from which new plants arise. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile.

The term “allele(s)” means any of one or more alternative forms of a gene at a particular locus. In a diploid (or amphidiploid) cell of an organism, alleles of a given gene are located at a specific location or locus on a chromosome, with one allele being present on each chromosome of the pair of homologous chromosomes. Similarly, in a tetraploid cell of an organism, one allele is present on each chromosome of the group of four homologous chromosomes. “Heterozygous” alleles are different alleles residing at a specific locus, positioned individually on corresponding homologous chromosomes. “Homozygous” alleles are identical alleles residing at a specific locus, positioned individually on corresponding homologous chromosomes in the cell.

“Wild type” as used herein refers to a typical form of a plant or a gene as it most commonly occurs in nature. A “wild type PPO allele” is a naturally occurring PPO allele (e.g., as found within naturally occurring S. tuberosum plants) that encodes a functional PPO protein, while a “non-functional mutant PPO allele” is a PPO allele that does not encode a functional PPO protein. Such a “non-functional mutant PPO allele” can include one or more mutations in its nucleic acid sequence, where the mutation(s) result in reduced or even no detectable amount of functional PPO protein in the plant or plant cell in vivo.

“Mutagenesis” as used herein refers to processes in which mutations are introduced into a selected DNA sequence. Mutations induced by endonucleases generally are obtained by a double-strand break, which can result in an insertion/deletion mutation (“indel”) that can be detected by deep-sequencing analysis. Such mutations can be deletions of several base pairs, including frameshift mutations, that can have the effect of inactivating the mutated allele. In the methods described herein, for example, mutagenesis occurs via double-stranded DNA breaks made by TALE-nucleases targeted to selected DNA sequences in a plant cell. Such mutagenesis results in “TALE-nuclease-induced mutations” (e.g., TALE-nuclease-induced knockouts) and reduced expression of the targeted gene. Following mutagenesis, plants can be regenerated from the treated cells using known techniques (e.g., planting seeds in accordance with conventional growing procedures, followed by self-pollination).

The term “expression” as used herein refers to the transcription of a particular nucleic acid sequence to produce sense or antisense RNA or mRNA, and/or the translation of an mRNA molecule to produce a polypeptide, with or without subsequent post-translational events.

“Reducing the expression” of a gene or polypeptide in a plant or a plant cell includes inhibiting, interrupting, knocking-out, or knocking-down the gene or polypeptide, such that transcription of the gene and/or translation of the encoded polypeptide is reduced as compared to a corresponding control plant or plant cell in which expression of the gene or polypeptide is not inhibited, interrupted, knocked-out, or knocked-down. “Reduced expression” encompasses any decrease in expression level (e.g., a decrease of 10% or more, 20% or more, 30% or more, 40% or more, 50% or more, 60% or more, 70% or more, 80% or more, 90% or more, or even 100%) as compared to the corresponding control plant, plant cell, or population of plants or plant cells. In some embodiments, reducing expression by 50% or more may be particularly useful. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

The potato genome contains multiple PPO genes, and because cultivated potato is a tetraploid, multiple alleles of each PPO gene are present in each variety. The methods provided herein can be used to inactivate at least one (e.g., at least two, at least three, or all four) functional alleles of one or more (e.g., one, two, three, four, five, or six) PPO genes, thereby removing at least some full-length RNA transcripts and functional PPO protein from potato cells, and in some cases completely removing all full-length RNA transcripts and functional PPO protein encoded by one or more given PPO genes.

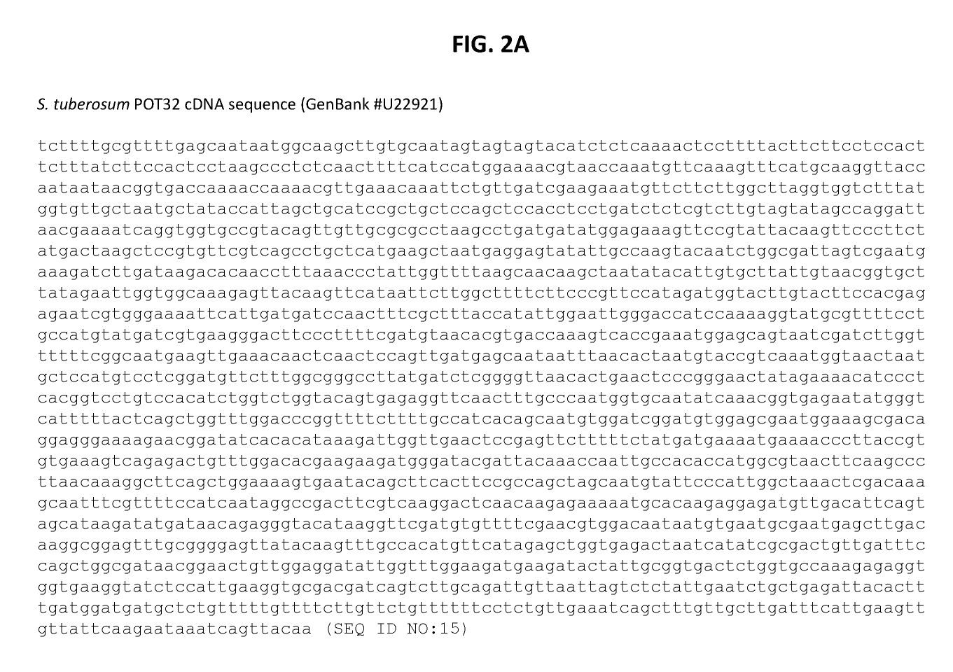

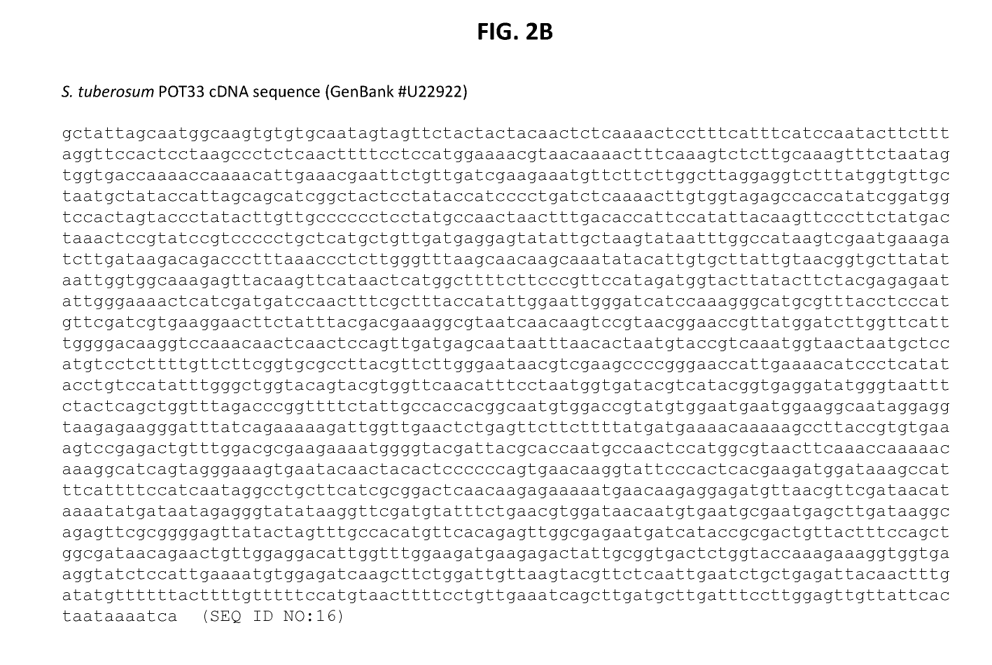

Representative examples of naturally occurring S. tuberosum tuber-specific expressed PPO nucleotide sequences (POT32 (SEQ ID NO:15) and POT33 (SEQ ID NO: 16)) are shown in FIGS. 2A and 2B, respectively. In some embodiments, the S. tuberosum plants, cells, plant parts, seeds, and progeny thereof that are provided herein can have a mutation in each endogenous allele of one or more PPO genes (e.g., each endogenous POT32 allele, each endogenous POT33 allele, or each endogenous POT32 and POT33 allele), such that expression of the gene is reduced or completely inhibited. Thus, in some cases, the plants, cells, plant parts, seeds, and progeny do not exhibit detectable levels of polyphenol oxidase expressed from the PPO genes.

The plants, plant cells, plant parts, seeds, and progeny provided herein can be generated using a rare-cutting endonuclease system to make a targeted knockout in one or more alleles of one or more PPO genes. Thus, this document provides materials and methods for using rare-cutting endonucleases (e.g., transcription activator-like effector endonucleases (TALE-nucleases)) to generate potato plants and related products (e.g., seeds and plant parts) that are particularly suitable for generating plant material with reduced PPO expression, due to targeted knockouts in the PPO genes. Other sequence-specific nucleases also may be used to generate the desired plant material, including engineered homing endonucleases or zinc finger nucleases (ZFNs).

The term “rare-cutting endonuclease” as used herein refers to a natural or engineered protein having endonuclease activity directed to a nucleic acid sequence with a recognition sequence (target sequence) about 12-40 bp in length (e.g., 14-40, 15-36, or 16-32 bp in length). See, e.g., Baker, Nature Methods 9:23-26, 2012. Typical rare-cutting endonucleases cause cleavage inside their recognition site, leaving 4 nt staggered cuts with 3′OH or 5′OH overhangs. In some embodiments, a rare-cutting endonuclease can be a meganuclease, such as a wild type or variant homing endonuclease (e.g., belonging to the dodecapeptide family (LAGLIDADG (SEQ ID NO: 17); see, WO 2004/067736). In some embodiments, a rare-cutting endonuclease can be a fusion protein containing a DNA binding domain and a catalytic domain with cleavage activity. TALE-nucleases and ZFNs are examples of fusions of DNA binding domains with the catalytic domain of the endonuclease FokI. Customized TALE-nucleases are commercially available under the trade name TALEN™ (Cellectis, Paris, France).

Transcription activator-like (TAL) effectors are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc. Natl. Acad. Sci. USA 103:10503-10508, 2006; Kay et al. Science 318:648-651, 2007; Sugio et al., Proc. Natl. Acad. Sci. USA 104:10720-10725, 2007; and Romer et al. Science 318:645-648, 2007). Specificity depends on an effector-variable number of imperfect, typically 34 amino acid repeats (Schornack et al., J. Plant Physiol. 163:256-272, 2006; and WO 2011/072246). Polymorphisms are present primarily at repeat positions 12 and 13, which are referred to herein as the repeat variable-diresidue (RVD).

The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

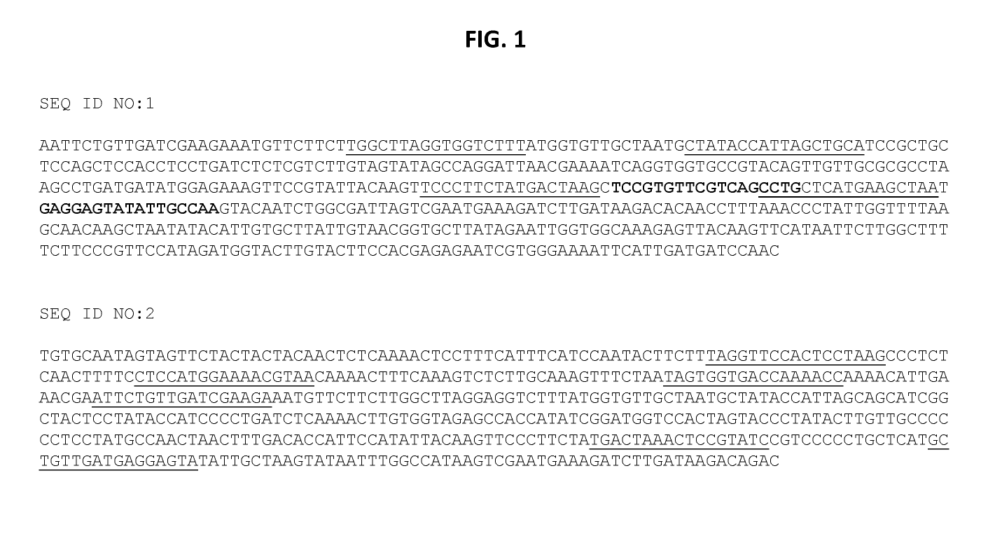

TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (double-stranded breaks) in DNA can induce mutations into the wild type DNA sequence via NHEJ or homologous recombination, for example. In some cases, TALE-nucleases can be used to facilitate site-directed mutagenesis in complex genomes, knocking out or otherwise altering gene function with great precision and high efficiency. As described in the Examples below, TALE-nucleases targeted to the S. tuberosum PPO genes can be used to mutagenize the endogenous gene, resulting in plants without detectable tuber expression of PPO. The fact that some endonucleases (e.g., FokI) function as dimers can be used to enhance the target specificity of the TALE-nuclease. For example, in some cases a pair of TALE-nuclease monomers targeted to different DNA sequences (e.g., the underlined target sequences shown in FIG. 1) can be used. When the two TALE-nuclease recognition sites are in close proximity, as depicted in FIG. 1, the inactive monomers can come together to create a functional enzyme that cleaves the DNA. By requiring DNA binding to activate the nuclease, a highly site-specific restriction enzyme can be created.

In general, a mutated Solanum plant, plant part, or plant cell as provided herein can have its expression of PPO reduced by 50 percent or more (e.g., by 60 percent or more, 70 percent or more, 80 percent or more, or 90 percent or more) as compared to a control Solanum plant that lacks the mutation(s). Further, the level of quinones in a mutated Solanum plant as provide herein can be decreased by about 50 percent or more (e.g., by 60 percent or more, 70 percent or more, 80 percent or more, or 90 percent or more) as compared to the control Solanum plant or population of Solanum plants. Methods for measuring quinone levels in plants are known in the art. See, e.g., Rauwald, H W, Pharm. Ztg. Wiss. 135:169-181, 1990. The control Solanum plant can be, for example, a corresponding wild-type version of the Solanum plant in which the PPO gene(s) were mutated.

In some cases, a Solanum plant can contain a PPO nucleotide sequence with at least 75 percent sequence identity to a representative PPO nucleotide sequence. For example, a nucleotide sequence can have at least 75 percent, at least 80 percent, at least 85 percent, at least 90 percent, at least 91 percent, at least 92 percent, at least 93 percent, at least 94 percent, at least 95 percent, at least 96 percent, at least 97 percent, at least 98 percent, or at least 99 percent sequence identity to a representative, naturally occurring PPO nucleotide sequence (e.g., SEQ ID NO:1, 2, 15, or 16).

In some cases, a mutation can be at a target sequence as set forth in a PPO sequence as set forth herein (e.g., SEQ ID NO: 1 or SEQ ID NO:2), or at a target sequence that is at least 95 percent (e.g., at least 96 percent, at least 97 percent, at least 98 percent, or at least 99 percent) identical to the sequence set forth in a PPO sequence as set forth herein (e.g., SEQ ID NO:1 and SEQ ID NO:2).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q −1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 100 consecutive nucleotides or amino acid residues from a sequence set forth in an identified sequence), followed by multiplying the resulting value by 100. For example, a nucleic acid sequence that has 480 matches when aligned with the sequence set forth in SEQ ID NO:1 is 96.0 percent identical to the sequence set forth in SEQ ID NO:1 (i.e., 480÷500×100=96.0). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Methods for selecting endogenous target sequences and generating TALE-nucleases targeted to such sequences can be performed as described elsewhere. See, for example, PCT Publication No. WO 2011/072246, which is incorporated herein by reference in its entirety. In some embodiments, software that specifically identifies TALE-nuclease recognition sites, such as TALE-NT 2.0 (Doyle et al., Nucleic Acids Res 40:W117-122, 2012) can be used.

Methods for using rare-cutting endonucleases (e.g., TALE-nucleases) to generate potato plants, plant cells, or plant parts having mutations in endogenous genes include, for example, those described in the Examples herein. For example, one or more nucleic acids encoding TALE-nucleases targeted to selected PPO sequences (e.g., the PPO sequences shown in FIG. 1) can be transformed into plant cells (e.g., protoplasts), where they can be expressed. In some cases, one or more TALE-nuclease proteins can be introduced into plant cells (e.g., protoplasts). The cells, or a plant cell line or plant part generated from the cells, can subsequently be analyzed to determine whether mutations have been introduced at the target site(s), through nucleic acid-based assays or protein-based assays to detect expression levels as described above, for example, or using nucleic acid-based assays (e.g., PCR and DNA sequencing, or PCR followed by a T7E1 assay; Mussolino et al., Nucleic Acids Res. 39:9283-9293, 2011) to detect mutations at the genomic loci. In a T7E1 assay, genomic DNA can be isolated from pooled calli, and sequences flanking TALE-nuclease recognition sites for PPO can be PCR-amplified. Amplification products then can be denatured and re-annealed. If the re-annealed fragments form a heteroduplex, T7 endonuclease I cuts at the site of mismatch. The digested products can be visualized by gel electrophoresis to quantify mutagenesis activity of the TALE-nuclease.

In some embodiments, a method as provided herein can include contacting a population of Solanum plant cells (e.g., protoplasts) having a functional PPO allele with a rare-cutting endonuclease that is targeted to an endogenous PPO sequence, selecting from the population a cell in which at least one (e.g., one, two, three, or four) PPO alleles have been inactivated, and growing the selected cell into a Solanum plant. The plant may have reduced levels of quinones as compared to a control Solanum plant that does contain the inactivated PPO allele(s). The rare-cutting endonuclease can be introduced into the population of cells via a nucleic acid (e.g., a vector or an mRNA) that encodes the rare-cutting endonuclease, or as a protein. In some cases, a method as provided herein can include a step of culturing a plant cell containing the inactivated PPO allele(s) to generate one or more plant lines. In addition or alternatively, a method as provided herein can include a step of isolating genomic DNA containing at least a portion of the PPO locus from the plant cells.

In some embodiments, a nuclease can be co-delivered with a plasmid encoding one or more exonuclease proteins to increase sequence specific nuclease-induced mutagenesis efficiency. Such exonucleases include, without limitation, members of the Trex (Therapeutic red cell exchange exonucleases) family of exonucleases, such as TREX2 (Shevelev et al., Scientific World Journal 2:275-281, 2002). Co-delivery of an exonuclease such as TREX with one or more TALE-nucleases may increase the frequency of NHEJ events, as compared with the frequency of NHEJ events observed after delivery of TALE-nucleases alone. It is to be noted that other suitable exonucleases also can be used in the methods provided herein.

Another genome engineering tool that can be used in the methods provided herein is based on the RNA-guided Cas9 nuclease from the type II prokaryotic CRISPR (Clustered Regularly Interspaced Short palindromic Repeats) adaptive immune system (see, e.g., Belahj et al., Plant Methods 9:39, 2013). This system allows for cleavage of DNA sequences that are flanked by a short sequence motif, referred as proto-spacer adjacent motif (PAM). Cleavage is achieved by engineering a specific crRNA that is complementary to the target sequence. The crRNA associates into a living cell with a heterologously expressed Cas9 endonuclease from Streptococcus pyogenes. In the crRNA/Cas9 complex, a dual tracrRNA:crRNA structure acts as a guide RNA that directs the Cas9 endonuclease to the cognate target sequence. Since several PAM motifs are present in the nucleotide sequence of the PPO genes, crRNA specific to the PPO genes can be designed to introduce mutations or to inactivate one or more PPO alleles within Solanum plant cells into which the Cas9 endonuclease and the crRNA are transfected and then expressed. In some embodiments, therefore, this approach can be used to obtain PPO mutant plants as described herein.

In some embodiments, the Cas protein can be a “functional derivative” of a naturally occurring Cas protein. A “functional derivative” of a native (naturally occurring) polypeptide is a compound having a qualitative biological property in common with the native polypeptide. “Functional derivatives” include, but are not limited to, fragments of native polypeptide, derivatives of a native polypeptide, and derivatives of fragments of a native polypeptide, provided that the fragments and derivatives have a biological activity in common with the corresponding native polypeptide. A biological activity contemplated herein is, for example, the ability of the functional derivative to hydrolyze a DNA substrate into fragments. The term “derivative” encompasses amino acid sequence variants of a polypeptide, covalent modifications of a polypeptide, and polypeptide fusions. Suitable derivatives of a Cas polypeptide or a fragment thereof include, without limitation, mutants, fusions, covalently modified Cas polypeptides, and fragments thereof.

In some embodiments, the Cas protein can be a NmCas9, StCas9, or SaCas9 polypeptide (see, for example, Esvelt et al., Nat Methods 10:1116-1121, 2013; Steinert et al., Plant J 84:1295-1305; Kaya et al., Sci Rep 6:26871, 2016; Zhang et al., Sci Rep 7:41993, 2017; and Kaya et al., Plant Cell Physiol 58:643-649, 2017). In addition to Cas9, CRISPR systems from Prevotella and Francisella 1 (Cpf1) can be used in the methods provided herein (see, for example, Zetsche et al., Cell 163:759-771, 2015).

In some embodiments, the plants provided herein can contain further mutations introduced into other Solanum genes. Such mutations can, for example:
provide acrylamide reduction by modifying the expression of genes involved in asparagine synthesis;
prevent Potato Virus Y by reducing eIF4E gene expression;
prevent late blight; and/or
improve nematode, herbicide, or insect resistance.
   Thus, the methods provided herein can be used to obtain gene stacking in a Solanum trait.

Further, this document provides bruising resistant tubers that are generated from a Solanum plant or plant part containing a mutation in each PPO allele endogenous to the plant or plant part—such that the plant, plant part, or plant cell has no functional PPO allele.

In a further embodiment, this document provides a heat-processed product of the plant. The heat-processed product can be a French fry, chip, crisp, potato, dehydrated potato, or baked potato, for example.

The invention will be further described in the following examples, which do not limit the scope of the invention described in the claims.

### EXAMPLES

### Example 1—Engineering Sequence-Specific Nucleases to Mutagenize the Tuber Specific Expressed PPO Genes

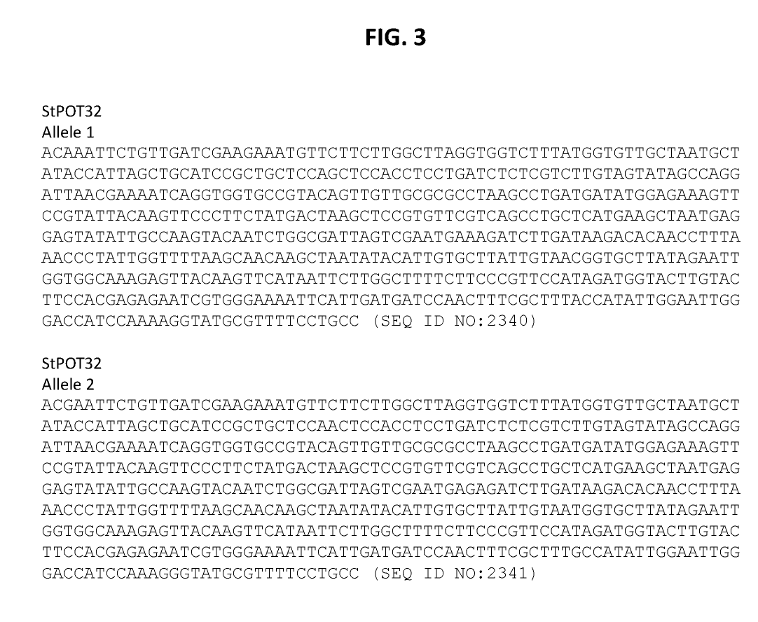

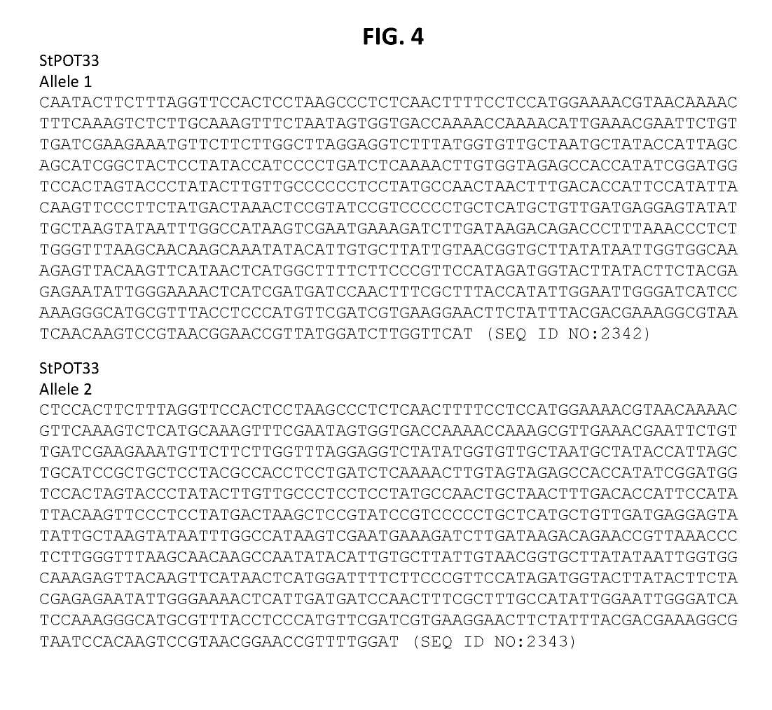

To identify potential TALE-nuclease target sites, the 5′ ends of the POT32 and POT33 genes were sequenced in Solanum tuberosum (Ranger Russet). Two different alleles were identified for both POT32 (SEQ ID NO:2340 and SEQ ID NO:2341) and POT33 (SEQ ID NO:2342 and SEQ ID NO:2343; see, FIGS. 3 and 4. Sequencing information was used to identify potential target sites for TALE-nucleases.

To completely inactivate or knock-out the alleles of the PPO genes in S. tuberosum, sequence-specific nucleases were designed to target the protein coding region in the first exon. TALE-nucleases were designed to target one or more tuber-specific expressed PPO genes, in particular POT32 and POT33, within the first 500 bp of the coding sequence, using software that specifically identifies TALE-nuclease recognition sites.

TALE-nuclease recognition sites for the POT32 and POT33 genes are underlined and bolded in FIG. 1, and are listed in TABLE 1 (SEQ ID NOS:3-14). TALE-nucleases were generated using methods similar to those described elsewhere (Cermak et al., Nucleic Acids Res. 39: e82, 2011; Reyon et al., Nat. Biotechnol. 30:460-465, 2012; and Zhang et al., Nat. Biotechnol. 29:149-153, 2011).

TABLE 1
TALE-nuclease target sequences

| | Target | | Target | |
|---|---|---|---|---|
| | Sequence | SEQ | Sequence | SEQ |
| Gene | Left | ID: | Right | ID: |
| POT32_T1 | TGGCTTAGG | 3 | CTATACCAT | 4 |
| | TGGTCTTT | | TAGCTGCA | |
| POT32_T2 | TCCCTTCTA | 5 | CCTGCTCAT | 6 |
| | TGACTAAG | | GAAGCTAA | |
| POT32_T3 | TCCGTGTTC | 7 | GAGGAGTAT | 8 |
| | GTCAGCCT | | ATTGCCAA | |
| POT33_T1 | TAGGTTCCA | 9 | CTCCATGGA | 10 |
| | CTCCTAAG | | AAACGTAA | |
| POT33_T2 | TAGTGGTGA | 11 | ATTCTGTTG | 12 |
| | CCAAAACC | | ATCGAAGA | |
| POT33_T3 | TGACTAAAC | 13 | GCTGTTGAT | 14 |
| | TCCGTATC | | GAGGAGTA | |

### Example 2—Activity of PPO TALE-Nucleases at their Endogenous Target Sites in S. tuberosum

TALE-nuclease activity at endogenous target sites in S. tuberosum was measured by expressing the TALE-nucleases in protoplasts and then surveying the TALE-nuclease target sites for mutations introduced by NHEJ. Methods for protoplast preparation were performed as described elsewhere (Shepard, in: Genetic Improvement of Crops/Emergent Techniques, Rubenstein, Gengenbach, Philips, and Green (Eds.), Univ. of Minnesota Press, Minneapolis, Minn., 1980, pp. 185-219; and Shepard and Totten, Plant Physiol. 60:313-316, 1977). Briefly, S. tuberosum mini tubers were planted in moistened vermiculite and grown under low light conditions for 3-5 weeks. Young, fully expanded leaves were collected and surface sterilized, and protoplasts were isolated.

TALE-nuclease-encoding plasmids, together with a yellow fluorescent protein- (YFP-) encoding plasmid, were introduced into S. tuberosum protoplasts by polyethylene glycol- (PEG-) mediated transformation as described elsewhere (Yoo et al., Nature Protocols 2:1565-1572, 2007). In some cases, a plasmid encoding a TREX2 exonuclease was co-delivered with the TALE-nuclease-encoding plasmid. Twenty-four hours after treatment, transformation efficiency was measured using a fluorescent microscope to monitor YFP fluorescence in an aliquot of the transformed protoplasts. The remainder of the transformed protoplasts was harvested, and genomic DNA was prepared using a hexadecyltrimethylammonium bromide-(CTAB-) based method. Using genomic DNA prepared from the protoplasts as a template, a DNA fragment encompassing the TALE-nuclease recognition site was amplified by PCR. Allele types were analyzed by individual clonal direct sequencing and 454 pyro-sequencing. Sequencing reads with indel mutations in the spacer region were considered to be derived from imprecise repair of a cleaved TALE-nuclease recognition site by NHEJ. Mutagenesis frequency was calculated as the number of sequencing reads with NHEJ mutations out of the total sequencing reads.

Primers used to amplify the TALE-nuclease target sites within the POT32 and POT33 genes are shown in TABLE 2.

TABLE 2
Pruner sequences for amplifying
POT32 and POT33 TALE target sequences

| Primer | | | SEQ |
|---|---|---|---|
| pair | | Forward Primer Sequence | ID: |
| POT32 | Forward | GCAAGCTTGTGCAATAGTAGTAG | 18 |
| | Reverse | AACCAATAGGGTTTAAAGGTTG | 19 |
| POT33 | Forward | CAAGTGTGTGCAATAGTAGTTCTAC | 20 |
| | Reverse | CCAAGAGGGTTTAAAGGGTC | 21 |

Sequences containing mutations introduced by TALE nuclease pair POT32_T1 within the POT32 alleles are set forth in SEQ ID NOs:22-112, with sequences containing deletions introduced by TALE nuclease pair POT32_T1 set forth in SEQ ID NOs:22-96, and insertions introduced by TALE nuclease pair POT32_T1 set forth in SEQ ID NOs:97-112.

Sequences containing mutations introduced by TALE nuclease pair POT32_T2 within the POT32 alleles are set forth in SEQ ID NOs: 113-711. In particular, sequences containing deletions introduced by TALE nuclease pair POT32_T2 are set forth in SEQ ID NOs:113-600, and sequences containing insertions introduced by TALE nuclease pair POT32_T2 are set forth in SEQ ID NOs:601-711.

Sequences containing mutations introduced by TALE nuclease pair POT32_T03 within the POT32 alleles are set forth in SEQ ID NOs:712-1228. Specifically, sequences containing deletions introduced by TALE nuclease pair POT32_T3 are set forth in SEQ ID NOs:712-1171, and sequences containing insertions introduced by TALE nuclease pair POT32_T3 are set forth in SEQ ID NOs:1172-1228.

Sequences containing mutations introduced by TALE nuclease pair POT33_T1 within the POT33 alleles are set forth in SEQ ID NOs:1229-1514, with sequences containing deletions introduced by TALE nuclease pair POT33_T1 set forth in SEQ ID NOs: 1229-1475, and sequences containing insertions introduced by TALE nuclease pair POT33_T1 set forth in SEQ ID NOs:1476-1514.

Sequences containing mutations introduced by TALE nuclease pair POT33_T2 within the POT33 alleles are set forth in SEQ ID NOs:1515-1953. Specifically, sequences containing deletions introduced by TALE nuclease pair POT33_T2 are set forth in SEQ ID NOs:1515-1857, and sequences containing insertions introduced by TALE nuclease pair POT33_T2 are set forth in SEQ ID NOs:1858-1953.

Sequences containing mutations introduced by TALE nuclease pair POT33_T3 within the POT33 alleles are set forth in SEQ ID NOs: 1954-2330. In particular, sequences containing deletions introduced by TALE nuclease pair POT33_T3 are set forth in SEQ ID NOs:1954-2231, and sequences containing insertions introduced by TALE nuclease pair POT33_T3 are set forth in SEQ ID NOs:2232-2330.

### Example 3—Regenerating S. tuberosum Lines with TALE-Nuclease-Induced PPO Mutations

S. tuberosum lines were created with mutations in one or more alleles of POT32 and POT33. Protoplasts isolated from surface sterilized leaves were transformed with plasmids encoding the following: (i) TALE-nuclease PPO_Tx or (ii) TALE-nuclease PPO_Tx+TREX2. Transformation efficiencies were monitored by delivery of a YFP plasmid, which was visualized using fluorescence microscopy or flow cytometry.

After PEG-mediated transformation, protoplasts are cultured using methods and media described elsewhere (Gamborg et al., in: Plant Tissue Culture Methods and Applications in Agriculture (pp. 115-153), Thorpe (Ed.), Academic Press, Inc., New York, N.Y., 1981), with slight modifications. Protoplasts are re-suspended in liquid plating medium at a cell density of 1×10⁵/ml in a small Petri dish, and stored at 25° C. in the dark. At 14 days post transformation (dpt), when the majority of the protoplasts have divided at least once, the protoplast culture is diluted two-fold in a suspension of P.-medium. At 28 dpt, the protoplast cultures are plated on a solid reservoir (10 ml) of CUL medium (Haberlach et al., Plant Science 39:67-74, 1985). At this point, protoplast-derived calli are visible to the eye.

At 65 dpt, protoplast-derived calli identified as mutants are transferred to a solid reservoir of DIF medium (Haberlach et al., supra). Calli are transferred to fresh DIF medium at biweekly intervals. As shoots form, they are excised and placed into a solid reservoir of R.-medium (Gamborg et al., supra). These individual calli are transferred to shoot-inducing medium. Once roots form, they are transferred to soil and grown to maturity for tuber production.

### Example 4—Verifying S. tuberosum Lines with TALE-Nuclease-Induced PPO Mutations

Once the protoplast-derived S. tuberosum lines were regenerated to plantlets, all alleles of the PPO genes were assessed for mutations. Plants with putative mutations in the PPO gene were verified by PCR amplification of the target locus, and subsequently sequenced.

Two mutant S. tuberosum plants were identified from protoplasts transformed with TALE-nuclease pair POT32_T3. The mutant plants are referred to as St381 and St465.

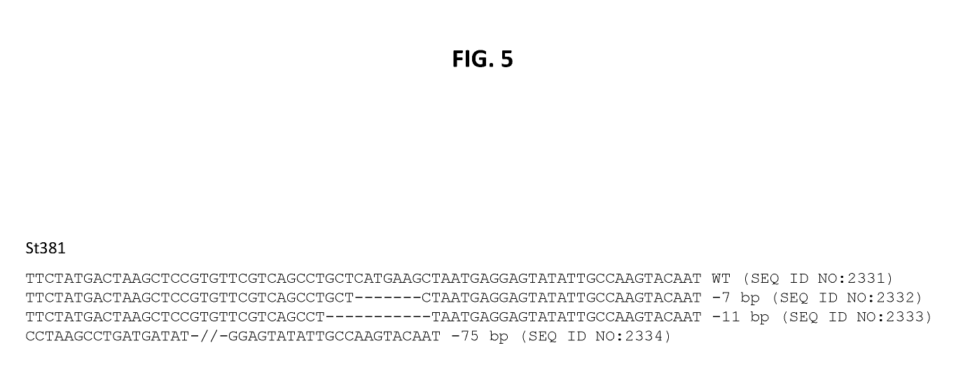

Deletions identified in POT32 from plant St381 are shown in FIG. 5. The mutations included a 7 bp deletion (SEQ ID NO:2331), an 11 bp deletion (SEQ ID NO:2333), and a 75 bp deletion (SEQ ID NO:2334).

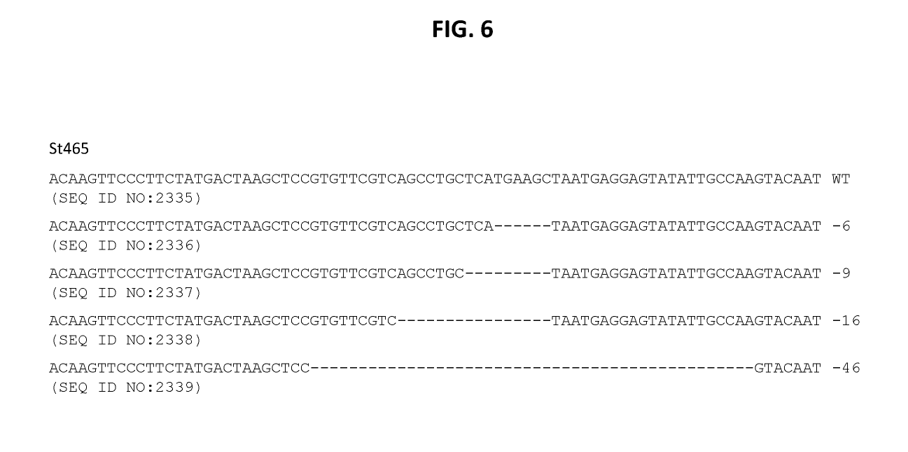

Deletions identified in POT32 from plant St465 are shown in FIG. 6. The mutations included a 6 bp deletion (SEQ ID NO:2336), a 9 bp deletion (SEQ ID NO:2337), a 16 bp deletion (SEQ ID NO:2338), and a 46 bp deletion (SEQ ID NO:2339).

### Example 5—Determining Whether Mutant S. tuberosum Lines have Desired Phenotypes

PPO transcript quantification is determined using quantitative real-time PCR of cDNA generated from mutant and control tuber mRNA extracts (Bhaskar et al., Plant Physiol. 154(2):939-948, 2010). The level and change (e.g., reduction) of PPO expression is quantified using the comparative cycle threshold method (Livak and Schmittgen, Method. Methods 25:402-408, 2001). Tuber-specific polyphenol oxidase levels is assessed using methods and media described elsewhere (Thygesen et al., supra).

For the phenotypic analysis, the relative polyphenolic-oxidase enzymatic activity was assayed. Tissue from potato tubers was homogenized by mortar and pestle and extracted in a cold, buffered detergent solution (100 mM NaP at pH 6.0, 2% TX-100, 2% PVP). Total protein content was quantified by Bradford Assay against a BSA standard curve. Extracts were added to a buffered solution of 4-methylcatechol (50 mM NaP at pH 6.0, 0.1% SDS, 15 mM 4-methylcatechol) and monitored for change in absorbance at 400 nm in 5 second intervals for 1.5 minutes. Relative reaction rate was determined by dividing the rate of absorbance change by the mg total protein added for each reaction.

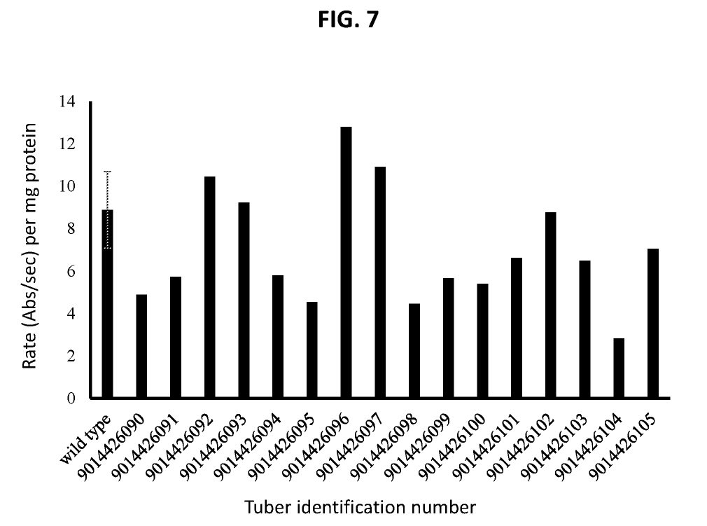

The polyphenolic-oxidase enzymatic activity assays indicated that tubers from St381 plants had different levels of polyphenolic-oxidase enzymatic activity than tubers from wild type plants (FIG. 7). Tubers from wild type plants had an observed polyphenolic-oxidase enzymatic activity [rate (Abs/sec) per mg protein] of 8.89. Four tubers from each of four different St381 plants were assayed. Tubers 9014426090-9014426093 from the first St381 plant had observed polyphenolic-oxidase enzymatic activities of 4.90, 5.73, 10.46, and 9.24. Tubers 9014426094-9014426097 from the second St381 plant had observed polyphenolic-oxidase enzymatic activities of 5.80, 4.54, 12.79, and 10.91. Tubers 9014426098-9014426101 from the third St381 plant had observed polyphenolic-oxidase enzymatic activities of 4.45, 5.66, 5.40, and 6.62. Tubers 9014426102-9014426105 from the fourth St381 plant had observed polyphenolic-oxidase enzymatic activities of 8.76, 6.49, 2.82, and 7.06.

These results indicated that TALE-nuclease-induced mutations within the POT32 gene can result in potato tubers with reduced polyphenolic-oxidase activity, as compared to potato tubers from unmodified plants.

### Example 6—POT TALE-Nucleases Targeting Copper-Binding Regions

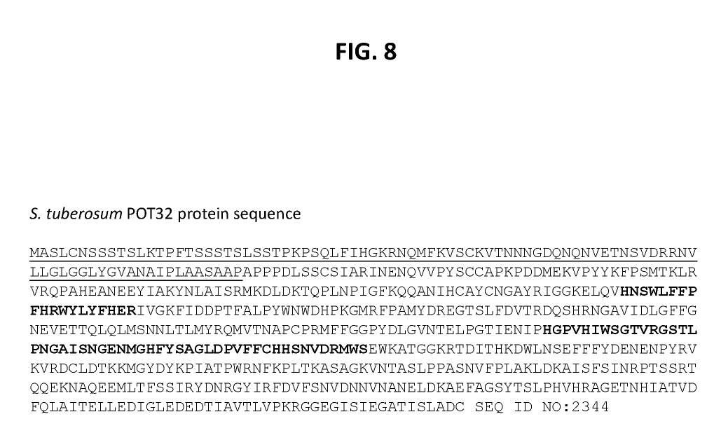

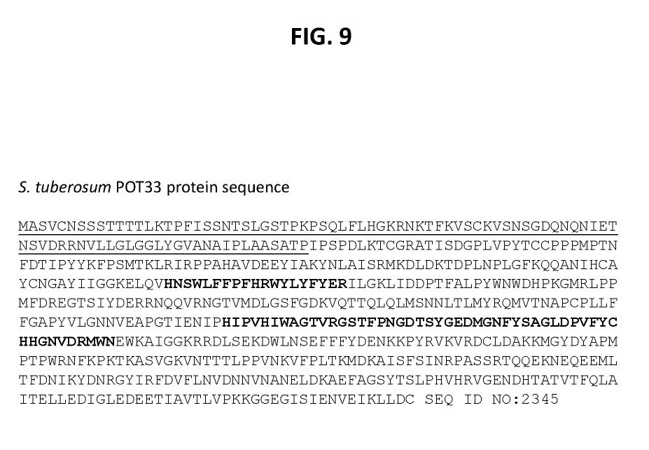

Polyphenol oxidase enzymes proteins are a group of copper-binding proteins that are widely distributed phylogenetically from bacteria to mammals, and that catalyze the oxidation of phenolics to quinones and produce brown pigments in wounded tissues. The amino acid sequence of POT32 is shown in FIG. 8 (SEQ ID NO:2344). The amino acid sequence of POT33 is shown in FIG. 9 (SEQ ID NO:2345). Both POT proteins have putative plastid transit peptide sequences at their N-termini, and contain two copper-binding regions, CuA and CuB. Additional TALE-nucleases can be designed to reduce PPO expression by producing in-frame or frameshift mutations within the POT32 and POT33 copper-binding regions. A list of additional TALE-nuclease target sites is shown in TABLE 3.

TABLE 3
TALE-nuclease target sequences

| | | Target | | Target | |
|---|---|---|---|---|---|
| | | sequence | SEQ | sequence | SEQ |
| Gene | Region | left | ID: | right | ID: |
| POT32 | CuA | TCTTGGCTT | 2346 | CTTGTACTT | 2347 |
| | | TTCTTCCC | | CCACGAGA | |
| POT32 | CuA | TTGGCTTTT | 2348 | TGTACTTCC | 2349 |
| | | CTTCCCGT | | ACGAGAGA | |
| POT32 | CuB | TGGTCTGGT | 2350 | CAATGGTGC | 2351 |
| | | ACAGTGAG | | AATATCAA | |
| POT33 | CuA | TGGCTTTTC | 2352 | TATACTTCT | 2353 |
| | | TTCCCGTT | | ACGAGAGA | |
| POT33 | CuB | TACCTGTCC | 2354 | GGTTCAACA | 2355 |
| | | ATATTTGG | | TTTCCTAA | |
| POT33 | CuB | TTGGGCTGG | 2356 | CTAATGGTG | 2357 |
| | | TACAGTAC | | ATACGTCA | |
| POT33 | CuB | TCCTAATGG | 2358 | TGGGTAATT | 2359 |
| | | TGATACGT | | TCTACTCA | |
| POT33 | CuB | TGGGTAATT | 2360 | GTTTTCTAT | 2361 |
| | | TCTACTCA | | TGCCACCA | |
| POT33 | CuB | TAGACCCGG | 2362 | GTGGACCGT | 2363 |
| | | TTTTCTAT | | ATGTGGAA | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A Solanum plant, plant part, or plant cell comprising a mutation in at least one polyphenol oxidase (PPO) allele endogenous to said plant, plant part, or plant cell, such that said plant, plant part, or plant cell has reduced expression of PPO as compared to a control Solanum plant, plant part, or plant cell that lacks said mutation.

2. The plant, plant part, or plant cell of claim 1, wherein each said mutation is a deletion of more than one nucleotide base pair.

3. The plant, plant part, or plant cell of claim 1, wherein each said mutation is at a target sequence as set forth in SEQ ID NO:2340 or 2341, or at a target sequence having at least 95 percent identity to SEQ ID NO:2340 or 2341.

4. The plant, plant part, or plant cell of claim 1, wherein each said mutation is at a target sequence as set forth in SEQ ID NO:2342 or 2343, or at a target sequence having at least 95 percent identity to SEQ ID NO:2342 or 2343.

5. The plant, plant part, or plant cell of claim 1, wherein said mutation was made using a rare-cutting endonuclease.

6. The plant, plant part, or plant cell of claim 5, wherein said rare-cutting endonuclease is a transcription activator-like effector endonuclease (TALE-nuclease).

7. The plant, plant part, or plant cell of claim 6, wherein said TALE-nuclease binds to a sequence as set forth in any of SEQ ID NOS:3-14 or 2346-2363.

8. The plant, plant part, or plant cell of claim 1, wherein said at least one PPO allele exhibits removal of an endogenous nucleic acid and does not include any exogenous nucleic acid.

9. The plant, plant part, or plant cell of claim 1, wherein said at least one PPO allele is a POT32 allele or a POT33 allele.

10. The plant, plant part, or plant cell of claim 9, wherein said at least one PPO allele is one or more POT32 alleles, and wherein said one or more POT32 alleles comprise one or more sequences selected from the group consisting of SEQ ID NO:2332, SEQ ID NO:2333, and SEQ ID NO:2334.

11. The plant, plant part, or plant cell of claim 9, wherein said at least one PPO allele is one or more POT32 alleles, and wherein said one or more POT32 alleles comprise one or more sequences selected from the group consisting of SEQ ID NO:2336, SEQ ID NO:2337, SEQ ID NO:2338, and SEQ ID NO:2339.

12. The plant, plant part, or plant cell of claim 9, wherein every endogenous allele of POT32 is mutated, every endogenous allele of POT33 is mutated, or every endogenous allele of both POT32 and POT33 is mutated.

13. The plant, plant part, or plant cell of claim 12, wherein each said POT32 allele, each said POT33 allele, or each said POT32 and POT33 allele exhibits removal of an endogenous nucleic acid and does not include any exogenous nucleic acid.

14. The plant, plant part, or plant cell of claim 1, wherein said plant, plant part, or plant cell has no detectable expression of PPO.

15. The plant, plant part, or plant cell of claim 1, wherein said Solanum plant, plant part, or plant cell is a Solanum tuberosum plant, plant part, or plant cell.

16. The plant, plant part, or plant cell of claim 1, wherein said plant, plant part, or plant cell has decreased levels of quinones as compared to a control plant, plant part, or plant cell that lacks said mutation.

17. A method for making a Solanum plant that has reduced expression of PPO, wherein said method comprises:
(a) contacting a population of Solanum plant cells comprising at least one functional endogenous PPO allele with one or more rare-cutting endonucleases targeted to said at least one PPO allele,
(b) selecting, from said population, a cell in which said at least one PPO allele has been mutated, and
(c) growing said selected plant cell into a Solanum plant, wherein said Solanum plant has reduced expression of PPO as compared to a control Solanum plant in which said at least one PPO allele has not been mutated.

18. The method of claim 17, wherein said Solanum plant cells are protoplasts.

19. The method of claim 18, comprising transforming said protoplasts with a nucleic acid encoding said rare-cutting endonuclease.

20. The method of claim 19, wherein said nucleic acid is an mRNA.

21. The method of claim 19, wherein said nucleic acid is contained within a vector.

22. The method of claim 18, comprising introducing into said protoplasts a rare-cutting endonuclease protein.

23. The method of claim 17, wherein said one or more rare-cutting endonucleases comprise a TALE-nuclease.

24. The method of claim 23, wherein said TALE-nuclease is targeted to a sequence as set forth in SEQ ID NO: 2340 or 2341, or to a sequence having at least 95 percent identity to the sequence set forth in SEQ ID NO: 2340 or 2341.

25. The method of claim 23, wherein said TALE-nuclease is targeted to a sequence as set forth in SEQ ID NO: 2342 or 2343, or to a sequence having at least 95 percent identity to the sequence set forth in SEQ ID NO: 2342 or 2343.

26. The method of claim 23, wherein said TALE-nuclease binds to a sequence as set forth in any of SEQ ID NOS:3-14 or 2346-2363.

27. The method of claim 17, wherein said at least one PPO allele is a POT32 allele or a POT33 allele.

28. The method of claim 27, wherein said at least one PPO allele is one or more POT32 alleles, and wherein said one or more POT32 alleles comprise one or more sequences selected from the group consisting of SEQ ID NO:2332, SEQ ID NO:2333, and SEQ ID NO:2334.

29. The method of claim 27, wherein said at least one PPO allele is one or more POT32 alleles, and wherein said one or more POT32 alleles comprise one or more sequences selected from the group consisting of SEQ ID NO:2336, SEQ ID NO:2337, SEQ ID NO:2338, and SEQ ID NO:2339.

30. The method of claim 17, wherein said Solanum plant cells are protoplasts, and wherein said method further comprises culturing said protoplasts to generate plant lines.

31. The method of claim 17, wherein said Solanum plant cells are protoplasts, and wherein said method further comprises isolating genomic DNA comprising at least a portion of the PPO locus from said protoplasts.

32. The method of claim 17, wherein said Solanum plant cells are S. tuberosum plant cells.

33. A method for producing a bruising resistant potato product, comprising:
(a) providing a Solanum plant or plant part that comprises a mutation in at least one PPO allele endogenous to said plant or plant part, such that said plant, plant part, or plant cell has reduced expression of PPO as compared to a control Solanum plant or plant part that lacks said mutation; and
(b) producing a bruising resistant potato product from said plant or plant part.

34. The method of claim 33, wherein said mutation is at a target sequence as set forth in SEQ ID NO: 2340 or 2341, or at a target sequence having at least 95 percent identity to SEQ ID NO: 2340 or 2341.

35. The method of claim 33, wherein said mutation is at a target sequence as set forth in SEQ ID NO: 2342 or 2343, or at a target sequence having at least 95 percent identity to SEQ ID NO: 2342 or 2343.

36. The method of claim 33, wherein said mutation was made using a rare-cutting endonuclease.

37. The method of claim 36, wherein said rare-cutting endonuclease is a TALE-nuclease.

38. The method of claim 37, wherein said TALE-nuclease binds to a sequence as set forth in any of SEQ ID NOS: 3-14 or 2346-2363.

39. The method of claim 33, wherein said at least one PPO allele is a POT32 allele or a POT33 allele.

40. The method of claim 39, wherein said at least one PPO allele is one or more POT32 alleles, and wherein said one or more POT32 alleles comprise one or more sequences selected from the group consisting of SEQ ID NO:2332, SEQ ID NO:2333, and SEQ ID NO:2334.

41. The method of claim 39, wherein said at least one PPO allele is one or more POT32 alleles, and wherein said one or more POT32 alleles comprise one or more sequences selected from the group consisting of SEQ ID NO:2336, SEQ ID NO:2337, SEQ ID NO:2338, and SEQ ID NO:2339.

42. The method of claim 33, wherein said Solanum plant or plant part is a S. tuberosum plant or plant part.

43. The method of claim 33, wherein said Solanum plant or plant part has no detectable expression of PPO.

44. A bruising resistant tuber generated from a Solanum plant or plant part that comprises a mutation in at least one PPO allele, such that said bruising resistant tuber has reduced expression of PPO as compared to a control Solanum plant, plant part, or plant cell that lacks said mutation.

45. The bruising resistant tuber of claim 44, wherein each said mutation is a deletion of more than one nucleotide base pair.

46. The bruising resistant tuber of claim 44, wherein each said mutation is at a target sequence as set forth in SEQ ID NO: 2340 or 2341, or at a target sequence having at least 95 percent identity to SEQ ID NO: 2340 or 2341.

47. The bruising resistant tuber of claim 44, wherein each said mutation is at a target sequence as set forth in SEQ ID NO: 2342 or 2343, or at a target sequence having at least 95 percent identity to SEQ ID NO: 2342 or 2343.

48. The bruising resistant tuber of claim 44, wherein each said mutation was made using a rare-cutting endonuclease.

49. The bruising resistant tuber of claim 48, wherein said rare-cutting endonuclease is a TALE-nuclease.

50. The bruising resistant tuber of claim 49, wherein said TALE-nuclease binds to a sequence as set forth in any of SEQ ID NOS:3-14 or 2346-2363.

51. The bruising resistant tuber of claim 44, wherein said tuber has decreased levels of quinones as compared to tubers generated from a control plant or plant part that lacks said mutation.

52. The bruising resistant tuber of claim 44, wherein said Solanum plant or plant part is a S. tuberosum plant or plant part.

53. The bruising resistant tuber of claim 44, wherein said at least one PPO allele is a POT32 allele or a POT33 allele.

54. The bruising resistant tuber of claim 53, wherein said at least one PPO allele is one or more POT32 alleles, and wherein said one or more POT32 alleles comprise one or more sequences selected from the group consisting of SEQ ID NO:2332, SEQ ID NO:2333, and SEQ ID NO:2334.

55. The bruising resistant tuber of claim 53, wherein said at least one PPO allele is one or more POT32 alleles, and wherein said one or more POT32 alleles comprise one or more sequences selected from the group consisting of SEQ ID NO:2336, SEQ ID NO:2337, SEQ ID NO:2338, and SEQ ID NO:2339.

56. A food product produced from the bruising resistant tuber of claim 44.

57. The food product of claim 56, wherein said food product has been cooked.

58. The food product of claim 57, wherein said food product is a French fry, chip, crisp, potato, dehydrated potato, or baked potato.
